(19) United States
(12) Patent Application Publication
(10) Pub. No.: US 2006/0122593 A1
(43) Pub. Date: Jun. 8, 2006

(54) Electrode for radiofrequency tissue ablation

(76) Inventor: Myong-Ki Jun, Kyunggi-Do (KR)

Correspondence Address: DARBY & DARBY P.C., P. O. BOX 5257, NEW YORK, NY, 10150-5257, US

(21) Appl. No.: 10/550,163

(22) PCT Filed: Apr. 22, 2004
(86) PCT No.: PCT/KR04/00933

§ 371 Date: Nov. 1, 2005

(30) Foreign Application Priority Data
Apr. 24, 2003 (KR) 10 2003-0026109

(51) Int. Cl. A61B 18/14

(52) U.S. Cl. 606/41

(57) ABSTRACT

The present invention discloses an electrode for an electric operation device including a hollow electrode being formed in a hollow tube shape extended long from a closed tip, and having an insulation-coating on the outside surface except a predetermined length of the closed tip side, a refrigerant tube having a smaller diameter than a diameter of the hollow electrode, and being inserted into the hollow electrode, the refrigerant tube supplying refrigerants for cooling a living tissue contacting the closed tip and the hollow electrode into the hollow electrode, and externally discharging the heat-exchanged refrigerants from the living tissue through the gap between the refrigerant tube and the hollow electrode, at least one first hole formed on the outside surface of the hollow electrode where the insulation coating has not been formed, for externally discharging some of the refrigerants supplied through the refrigerant tube from the hollow electrode, and a flow control means formed on the outside surface of the hollow electrode where the insulation coating has not been formed, and operated as a discharge resistance to the refrigerants discharged from the first hole, for controlling a flow of the refrigerants, whereby supplying an electrode structure using both a method for water-cooling the inside of the electrode and a method for discharging the saline solution.

### TECHNICAL FIELD

The present invention relates to an electrode for an electric operation device, and more particularly to, an electrode for an electric operation device for ablation and necrosis of a living tissue using RF electric energy.

### BACKGROUND ART

In general, a technique of ablating (or coagulating) a wanted living tissue with RF energy by inserting a long hollow tube-shaped electrode into the living tissue has been publicly known. When current is applied to the living tissue, the living tissue is heated, and thus the living tissue and blood vessels are ablated by a complicated biochemical process. This process depends on ablation of a cell by thermal transformation of cell proteins over about 60° C. Here, the cell implies the tissue, blood vessel and blood. However, the living tissue adjacent to the electrode and blood are excessively ablated and carbonized. The carbonized living tissue adjacent to the electrode is operated as an insulator, namely an obstacle to enlargement of an ablation zone of the living tissue.

In order to solve the above problem, U.S. Pat. No. 6,210,411 discloses a technique of supplying a saline solution though a hollow tube of an electrode, and externally discharging the saline solution through a porous sintered body formed at the tip of the electrode. As mentioned above, techniques of discharging a saline solution from an electrode prevent carbonization of living tissue adjacent to the electrode by a vaporization latent heat of the saline solution. In addition, the saline solution soaks into capillary vessels of the tissue adjacent to the electrode, to improve electric conductivity and enlarge the ablation zone of the living tissue. However, when a large volume of saline solution is infused into the living tissue, it has detrimental effects on patients. Therefore, the volume of the saline solution infused into the living tissue is restricted. When the RF e nergy applied to the living tissue exceeds a limit point, the tissue adjacent to the electrode is carbonized. As a result, the ablation zone is not efficiently enlarged.

In addition, U.S. Pat. No. 6,506,189 discloses a technique of installing a refrigerant tube having a smaller diameter than a diameter of a hollow tube-shaped electrode having a closed tip in the electrode, and cooling the electrode by refrigerant circulation of supplying refrigerants into the electrode through the refrigerant tube, exchanging heat in the electrode, and collecting the refrigerants through the gap between the refrigerant tube and the electrode. When the RF energy is applied by the electrode, the most adjacent tissue to the electrode is mostly heated and probably carbonized. Here, the most adjacent tissue contacting the electrode can be cooled by water-cooling the electrode, and thus prevented from being carbonized. Accordingly, the ablation zone of the living tissue can be enlarged. However, when the RF energy applied to the living tissue exceeds a limit point, the tissue adjacent to the electrode is carbonized. As a result, the ablation zone is not efficiently enlarged.

The aforementioned methods have been known to form a spherical ablation zone having a radius of about 2 cm from an electrode.

The conventional electrode for the electric operation device cools the living tissue adjacent to the electrode, by directly discharging the saline solution to the living tissue, or circulating the saline solution in the electrode. However, when the RF energy exceeding the limit point is generated, the tissue adjacent to the electrode is carbonized, and thus the ablation zone is not efficiently enlarged.

### DISCLOSURE OF THE INVENTION

The present invention is achieved to solve the above problems. An object of the present invention is to provide an electrode for an electric operation device which can enlarge an ablation zone of a living tissue and reduce an ablation and necrosis time of the living tissue, by supplying an electrode structure using both a method for cooling the inside of the electrode by a saline solution and a method for directly discharging the saline solution to the living tissue.

Another object of the present invention is to provide an electrode for an electric operation device which can enlarge an ablation zone of a living tissue and reduce an ablation and necrosis time of the living tissue, by supplying an electrode structure for gradually discharging some of a saline solution pressurized and infused into the electrode to the periphery of the living tissue.

In order to achieve the above-described objects of the invention, there is provided an electrode for an electric operation device, including: a hollow electrode being formed in a hollow tube shape extended long from a closed tip, and having an insulation-coating on the outside surface except a predetermined length of the closed tip side; a refrigerant tube having a smaller diameter than a diameter of the hollow electrode, and being inserted into the hollow electrode, the refrigerant tube supplying refrigerants for cooling a living tissue contacting the closed tip and the hollow electrode into the hollow electrode, and externally discharging the heat-exchanged refrigerants from the living tissue through the gap between the refrigerant tube and the hollow electrode; at least one first hole formed on the outside surface of the hollow electrode where the insulation coating has not been formed, for externally discharging some of the refrigerants supplied through the refrigerant tube from the hollow electrode; and a flow control means formed on the outside surface of the hollow electrode where the insulation coating has not been formed, and operated as a discharge resistance to the refrigerants discharged from the first hole, for controlling a flow of the refrigerants.

Preferably, the hollow electrode is conductive, and power is externally applied through the hollow electrode.

Preferably, the electrode for the electric operation device further includes a saline solution pipe being inserted onto the outside surface of the hollow electrode with a predetermined gap, and having an insulation coating on the outside surface except a predetermined length of the closed tip side, the saline solution pipe infusing a saline solution through the gap, and discharging the saline solution through at least one second hole formed on the outside surface where the insulation coating has not been formed. Here, the hollow electrode and the saline solution pipe are conductive, different power is applied to the hollow electrode and the saline solution pipe, and an insulation member for preventing short circuit by the saline solution supplied through the gap between the hollow electrode and the saline solution pipe is formed on the surface of the hollow electrode.

Preferably, the insulation member includes an insulation coating formed on the surface of the hollow electrode, and an insulation packing provided between the hollow electrode and the saline solution pipe.

More preferably, the closed tip of the hollow electrode is a conductive spearhead, and the hollow electrode and the spearhead are incorporated with each other.

Preferably, the flow control means is a hollow tube being inserted onto the outside surface of the hollow electrode where the insulation coating has not been formed, and having at least one third hole on the outside surface, the flow control means controlling a volume of the discharged refrigerants by alternately installing the first hole of the hollow electrode a and the third hole of the hollow tube, and operating as a discharge resistance to the refrigerants discharged from the first hole. More preferably, compression units of the hollow tube are formed in a zigzag shape on a discharge passage of the first hole, the third hole and both ends of the hollow tube, and operated as discharge resistances to the refrigerants discharged from the first hole, for controlling the volume of the discharged refrigerants.

Preferably, the flow control means is a porous metal sintered body layer formed on the outside surface of the hollow electrode where the insulation coating has not been formed, the sintered body layer being operated as a discharge resistance to the refrigerants discharged from the first hole, for controlling a volume of the discharged refrigerants.

### BRIEF DESCRIPTION OF THE DRAWINGS

The present invention will become better understood with reference to the accompanying drawings which are given only by way of illustration and thus are not limitative of the present invention, wherein:

FIG. 1A is a perspective view illustrating a first hole formed on the surface of a hollow electrode where an insulation coating has not been formed in an electrode for an electric operation device in accordance with one preferred embodiment of the present invention;

FIG. 1B is a perspective view illustrating a hollow tube having a third hole inserted onto the outside surface where the insulation coating has not been formed in the electrode for the electric operation device in accordance with the preferred embodiment of the present invention;

FIG. 2 is a perspective view illustrating disassembly of the electrode for the electric operation device in accordance with the preferred embodiment of the present invention;

FIG. 3 is a cross-sectional view illustrating the electrode for the electric operation device of FIG. 2;

FIG. 4 is a perspective view illustrating disassembly of an electrode for an electric operation device in accordance with another preferred embodiment of the present invention;

FIG. 5 is a cross-sectional view illustrating the electrode for the electric operation device of FIG. 4; and

FIGS. 6A and 6B are graphs showing RF power and current applied to the conventional electrode and the electrode of the invention, and impedance values of thermocouples installed therein, respectively.

### BEST MODE FOR CARRYING OUT THE INVENTION

An electrode for an electric operation device in accordance with the present invention will now be described in detail with reference to the accompanying drawings.

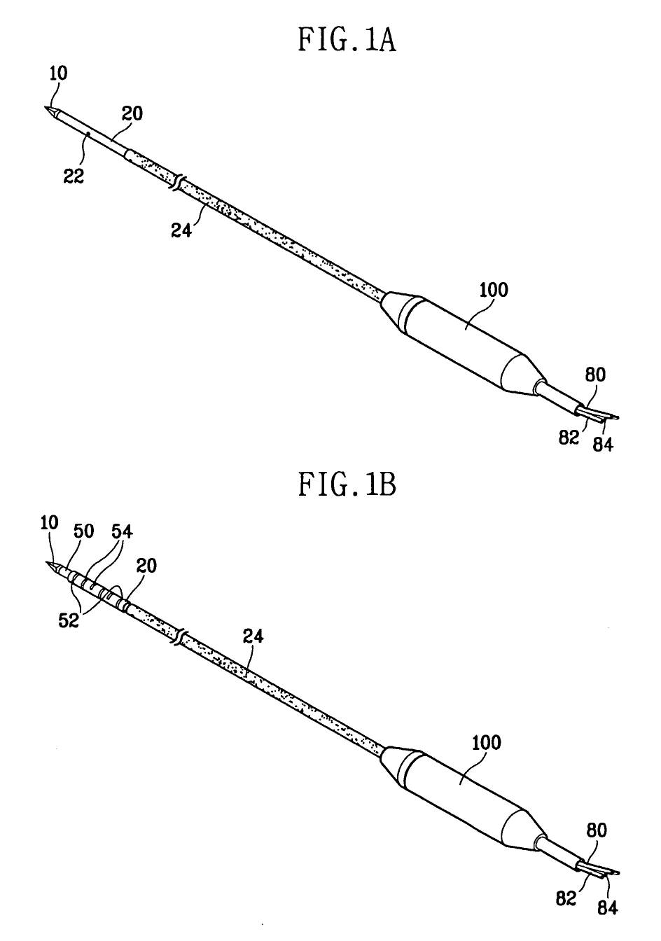

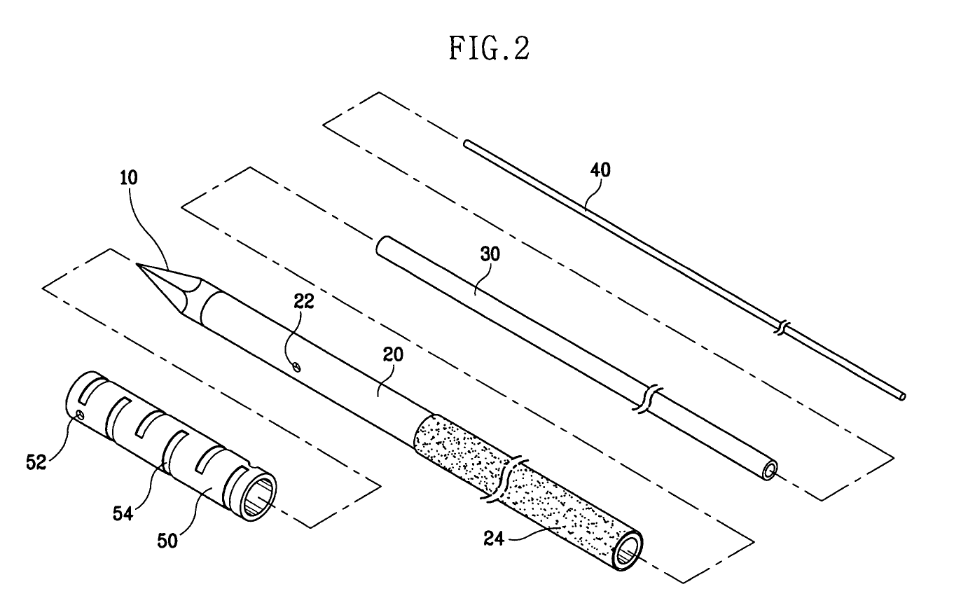

FIG. 1A illustrates an electrode for an electric operation device including a hollow electrode 20 having a refrigerant discharge hole 22 on the outside surface, and FIG. 1B illustrates a hollow tube 50 inserted onto the outside surface of the hollow electrode 20 and operated as a flow control means.

The electric operation device can be used in various application fields. For convenience' sake, it is exemplified that the electric operation device is applied to the operation of a patient suffering from a liver cancer.

A doctor inserts the electrode for the electric operation device as shown in FIGS. 1A and 1B into the body through the skin, moves the electrode for the electric operation device to a living tissue (for example, predetermined area of liver) for ablation and necrosis, supplies RF current from an external power source, and performs ablation and necrosis of the living tissue by the RF current in the tip 10 of the electrode for the electric operation device. Because an insulation coating 24 is formed on a large portion of the hollow electrode 20 by using an insulation material such as Teflon, ablation and necrosis are performed on the part of the hollow electrode 20 where the insulation coating 24 has not been formed and the periphery of the tip 10. As a result, ablation and necrosis are performed on the living tissue in a spherical shape. In this case, the living tissue contacting the hollow electrode 20 may be carbonized and operated as an insulator. It is thus very important to prevent carbonization of the living tissue to enlarge the ablation and necrosis zone.

In addition to the conventional technique of water-cooling the hollow electrode 20 and the living tissue contacting the hollow electrode 20 by supplying refrigerants into the hollow electrode 20, the present invention discharges some of the refrigerants into the living tissue where ablation and necrosis are being performed.

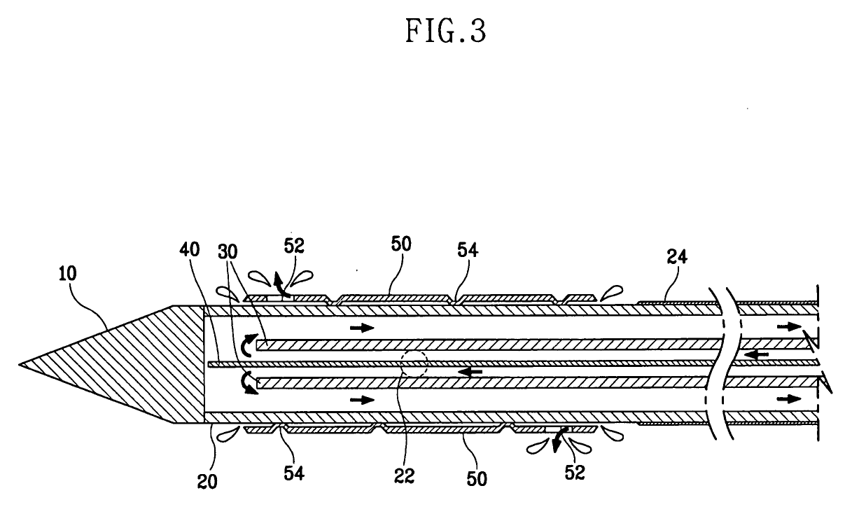

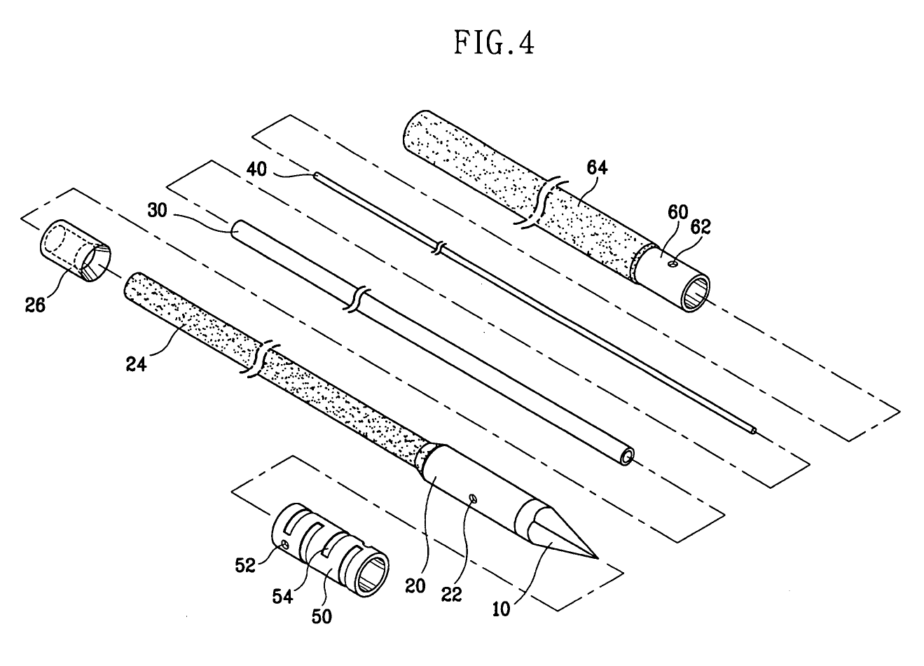

The refrigerants supplied into the hollow electrode 20 through a refrigerant tube 30 are infused into the hollow electrode 20 under a very high pressure (pressurized under approximately 700 to 1060KPa), for cooling the inside surface of the hollow electrode 20 and the tip 10, and discharged. FIGS. 2 and 3 illustrate the structure of the hollow electrode 20 and the refrigerant tube 30. An spearhead which is the tip 10 is incorporated with the hollow electrode 20. Here, the tip 10 is formed by using a conductive spearhead, and incorporated with the hollow electrode 20 by welding.

A large portion of the hollow electrode 20 is coated with the insulation coating 24. Even if the RF current is applied through the hollow electrode 20, the RF power is applied to the region where the insulation coating 24 has not been formed, but not applied to the other region. Reference numeral 40 denotes a temperature sensor line. The temperature sensor line 40 is inserted into the refrigerant tube 30, for sensing a temperature inside the tip 10 and the hollow electrode 20, and using the sensed temperature in the succeeding output control of the electrode.

In the electrode for the electric operation device, the refrigerants are internally supplied through the refrigerant tube 30 to exchange heat in the tip region of the hollow electrode 20, and externally discharged through the gap between the hollow electrode 20 and the refrigerant tube 30. FIGS. 1A and 1B show a supply tube 82 and a discharge tube 84. The refrigerants supplied through the supply tube 82 are internally transmitted through a handle 100 and the refrigerant tube 30. The heat-exchanged refrigerants are discharged from the body through the gap between the hollow electrode 20 and the refrigerant tube 30, and then discharged through the handle 100 and the discharge tube 84. In order to supply the refrigerants through the refrigerant tube 30 having a very small diameter of about 0.4mm, the pressure of the refrigerants must be very high as described above. Accordingly, still referring to FIG. 1A, when at least one first hole 22 is formed on the outside surface of the hollow electrode 20 where the insulation coating 24 has not been formed, even if a very small hole is formed by a mechanical process, the pressurized refrigerants are not prevented from being explosively spouted. The present invention provides a structure for efficiently discharging the pressurized refrigerants at a small amount in the electrode for the electric operation device for performing water-cooling by the pressurized refrigerants.

In this embodiment, a hollow tube 50 having a predetermined diameter so that the hollow tube 50 can be tightly inserted onto the hollow electrode 20 is used as a flow control means operated as a discharge resistance on a passage of the refrigerants discharged from the first hole 22 of the hollow electrode 20, for controlling a flow of the discharged refrigerants. The hollow tube 50 also includes at least one third hole 52 on the outside surface. Here, the first hole 22 formed on the hollow electrode 20 and the third hole 52 formed on the hollow tube 50 are alternately disposed. For example, the hollow tube 50 is inserted onto the hollow electrode 20 so that the first hole 22 and the third hole 52 can have an interval of 1800 from each other. In addition, the hollow tube 50 can be inserted onto the hollow electrode 20 so that the first holes 22 having an interval of 180° from each other and the third holes 52 having an interval of 1800 from each other can have an interval of 90 or 120° from each other. That is, the number and angle of the first hole 22 and the third hole 52 can be varied. Because the refrigerants are discharged into the living tissue, a physiological saline solution is preferably used. For example, 0.9% of saline solution, namely an isotonic solution can be used.

As schematically shown in FIG. 3, some of the refrigerants supplied through the refrigerant tube 30 and heat-exchanged are discharged from the first hole 22 formed on the outside surface of the hollow electrode 20. Because the hollow tube 50 is operated as a discharge resistance on the discharge passage, the refrigerants flow through the gap between the hollow tube 50 and the hollow electrode 20, and are discharged from the third hole 52 formed on the hollow tube 50. FIG. 3 shows discharge of the refrigerants in a state where the first and third holes 22 and 52 respectively formed at an interval of 180° are alternately disposed at an interval of 90° from each other. Still referring to FIG. 3, the refrigerants can be discharged through both ends of the hollow tube 50.

When compression units 54 are formed in a zigzag shape by press compression on the outside surface of the hollow tube 50 between the first hole 22 of the hollow electrode 20 and the third hole 52 of the hollow tube 50, the compression units 54 are operated as discharge resistances on the discharge passage, to efficiently control the flow of the refrigerants discharged under a high pressure. Each drawing shows the compression units 54. The refrigerants discharged from the first hole 22 are not directly discharged to the third hole 52 through the gap between the hollow tube 50 and the hollow electrode 20, but discharged to the third hole 52 via the compression units 54. To achieve better understanding, the size of each hole, the hollow tube 50 and the hollow electrode 20 is more exaggerated in each drawing.

When a filter or rib unit operated as a discharge resistance is formed in the hollow tube 50 and the hollow tube 50 is inserted onto the outside surface of the hollow electrode 20, such an additional member is operated as the discharge resistance on the discharge passage, to efficiently control the flow of the refrigerants discharged under a high pressure.

Although not illustrated, a porous metal sintered body layer comprised of a metal harmless to the human body can be formed on the portion of the hollow electrode 20 including the first hole 22 as the flow control means. In this case, even if a special third hole 52 is not formed on the porous metal sintered body layer, the porous metal sintered body layer is operated as a discharge resistance on the discharge passage. Therefore, the discharge flow can be efficiently controlled by adjusting the size and number of the first hole 22 and porosity of the porous metal sintered body layer.

The above-described electrode is a mono-polar electrode for forming a conductive hollow electrode, and externally supplying the RF power through the hollow electrode. Here, the electrode receiving the opposite polarity contacts the other part of the body.

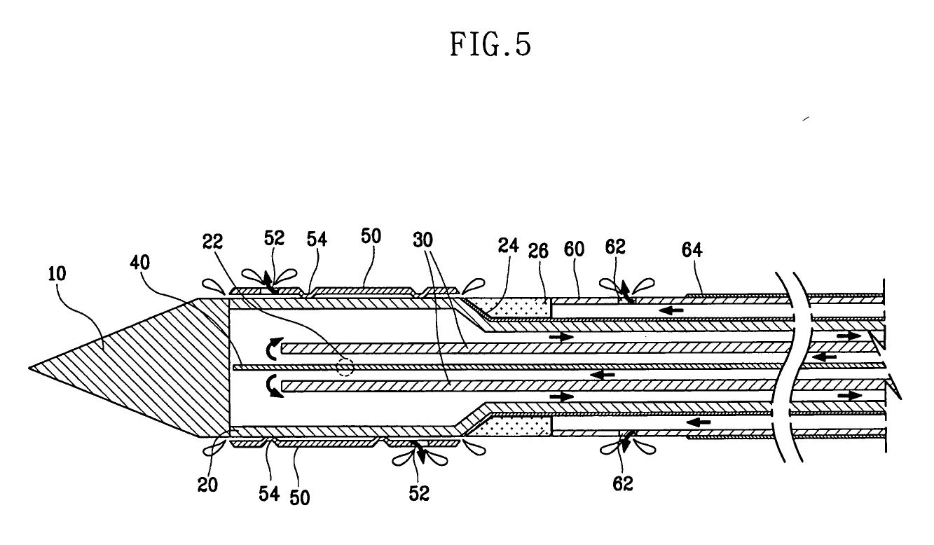

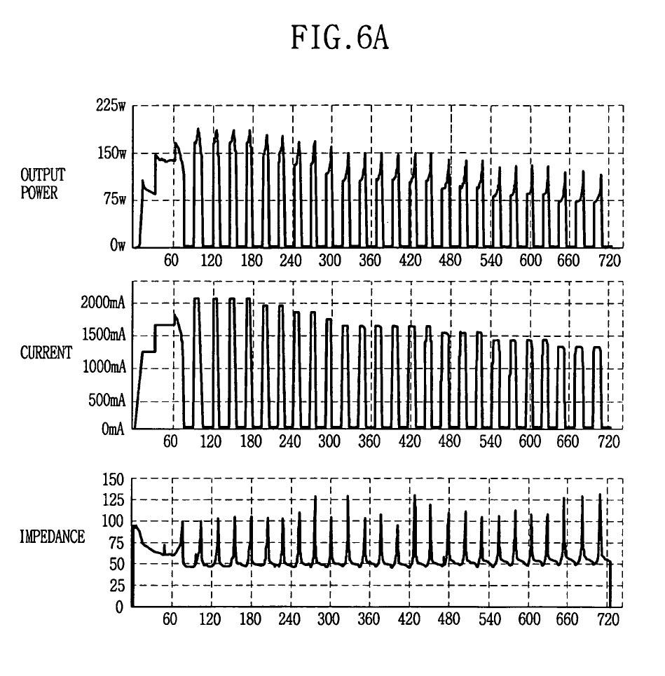

In accordance with another embodiment of the present invention, as shown in FIGS. 4 and 5, the electrode for the electric operation device further includes a saline solution pipe 60 inserted onto the outside surface of the hollow electrode 20 with a predetermined gap from the outside surface of the hollow electrode 20, for discharging a saline solution. As described above, the first hole 22 is formed in the tip side of the hollow electrode 20, and the hollow tube 50 is inserted onto the hollow electrode 20 so that the first hole 22 and the third hole 52 can be alternately disposed. In addition, the saline solution pipe 60 is inserted onto the outside surface of the hollow electrode 20, for supplying the saline solution through the gap between the inside surface of the saline solution pipe 60 and the outside surface of the hollow electrode 20 via a different tube from the refrigerant tube 30, and discharging the saline solution through a second hole 62 formed on the saline solution pipe 60. Here, an insulation coating is formed on a large portion of the saline solution pipe 60. In the previous embodiment, the refrigerants supplied through the refrigerant tube 30 are discharged through the flow control means, but in the current embodiment, the saline solution supplied through the saline solution pipe 60 is additionally discharged through the second hole 62. The saline solution supplied through the gap between the inside surface of the saline solution pipe 60 and the outside surface of the hollow electrode 20 has a relatively low pressure. Therefore, a volume of the saline solution discharged through the second hole 62 can be controlled by adjusting a supply volume thereof, without using a special flow control means.

Still referring to FIG. 4, a diameter of the hollow electrode 20 is maintained identical to that of the general hollow electrode 20 near the spearhead of the tip 10, and gets smaller after the first hole 22. Accordingly, a diameter of the saline solution pipe 60 is maintained equal or similar to that of the general hollow electrode 20. The electrode for the electric operation device is easily inserted into the living tissue, thereby minimizing pains or burdens of the patient. In this embodiment, the electrode can also be a mono-polar electrode for forming a conductive hollow electrode, and externally supplying the RF power through the hollow electrode. Here, the electrode receiving the opposite polarity contacts the other part of the body.

As illustrated in FIGS. 4 and 5, when the insulation coating 24 is formed on the surface of the hollow electrode 20 and an insulation packing 26 is formed, the electrode can be used as a bi-polar electrode. FIGS. 4 and 5 show the reduced diameter of the hollow electrode 20. Here, the diameter of the hollow electrode 20 is not essentially reduced. Most of all, it is important to remove short circuit between both electrodes in the bi-polar electrode. Here, power applied to the hollow electrode 20 is different from power applied to the saline solution pipe 60. Because the saline solution which is a conductor flows between the saline solution pipe 60 and the hollow electrode 20, short circuit is probably generated. Therefore, an insulation member must be provided to the portion of the hollow electrode 20 where the saline solution pipe 60 is inserted. In this embodiment, the insulation coating 24 is formed, and then the insulation packing 26 is formed to prevent short circuit from occurring when the saline solution is infused onto the hollow electrode 20 where the insulation coating 24 has not been formed through the gap between the saline solution pipe 60 and the insulation coating 24.

In this state, when different power is applied to the hollow electrode 20 and the saline solution pipe 60, ablation and necrosis of the living tissue are performed in the region where the insulation coating 24 has not been formed. The hollow electrode 20 near the tip 10 is water-cooled by the refrigerants pressurized and supplied through the refrigerant tube 30. Some of the refrigerants are discharged through the first hole 22, and externally discharged through the third hole 52 and/or both ends of the hollow tube 50 via the discharge resistances by the compression units 54 on the discharge passage. In addition, the saline solution is externally discharged through the second hole 62 of the saline solution pipe 60. As a result, the saline solution soaks into the living tissue and is operated as a conductor, to activate ablation and necrosis by the bi-polar electrode and enlarge the ablation and necrosis zone. FIG. 5 schematically shows discharge of the refrigerants and the saline solution.

### EXAMPLE

An experimental object was a cow liver, and an RF generator was a 480-kHz RF generator (Radionics, USA). 5.85% of saline solution was supplied in the electrode at a rate of 80 to 120 ml/min, and infused into the living tissue at a rate of 1 ml/cm. An ablation and necrosis experiment was executed 50 times by sequentially supplying the outputs of 30 sec-1.2 A (about 120 W), 30 sec-1.6 A (about 160 W) and 12˜15 sec-2 A (about 200 W), and maintaining an impedance between 50 and 110Ω. An ablation and necrosis zone was measured by the MRI.

Here, a thermocouple was embedded in the electrode. A temperature of the living tissue adjacent to the electrode was measured by an impedance of the thermocouple, and the RF power and current applied to the electrode were controlled according to the measured temperature, thereby preventing the living tissue from being excessively heated and carbonized, and performing ablation and necrosis on the living tissue in the wider zone.

Generally, the infusion amount of the saline solution allowed in the electric operation was about 120 cc/hr. The infusion amount of the saline solution in the experiment shorter than 15 minutes was 15 to 30 ml, which satisfied the standard.

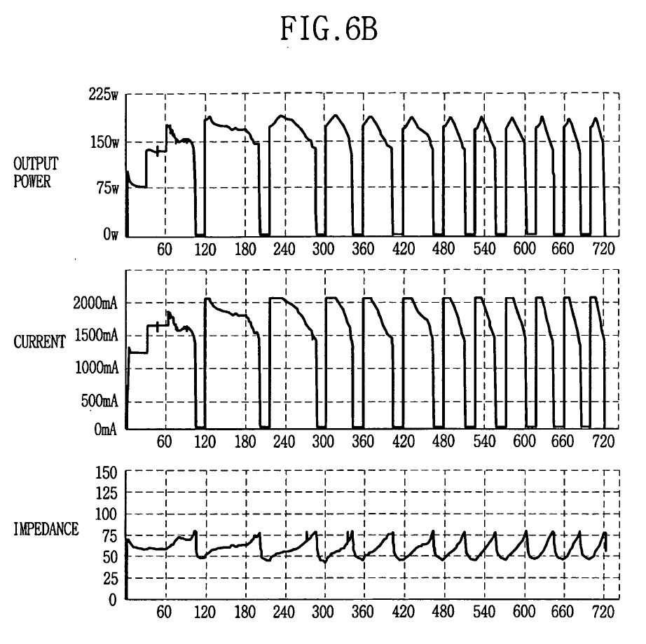

According to the experimental process using the conventional electrode for cooling the periphery of the electrode by circulating the coolant in the electrode, as shown in FIG. 6A, the impedance of the thermocouple sharply increases, and thus the maximum average impedance is 114.5±1.6. Therefore, the RF power, and current can be applied for 357±17 seconds.

Conversely, according to the experimental process using the electrode of the invention for cooling the periphery of the electrode by circulating the coolant in the electrode and directly discharging some of the coolant to the living tissue, as shown in FIG. 6B, the impedance of the thermocouple gradually increases, and thus the maximum average impedance is 83.5±4.4. Accordingly, the RF power and current can be applied for 540±18 seconds.

That is, the electrode of the invention shows higher cooling efficiency than the conventional electrode. Ablation and necrosis can be performed on part of the wanted living tissue for a short time, by gradually increasing the temperature of the living tissue adjacent to the electrode, and supplying the RF power and current for a long time.

When the experiment was performed under the aforementioned conditions, in the case of the conventional electrode, the minimum diameter, the maximum diameter and the volume of the ablation and necrosis zone were 3.6±0.34 cm, 4.1+0.38 cm and 23.1+8.7 cm³, and in the case of the electrode of the invention, the minimum diameter, the maximum diameter and the volume of the ablation and necrosis zone were 5.3+0.7 cm, 5.7±0.61 cm and 80±34 cm³. The radius of the method using the electrode of the invention increased more than that of the method using the conventional electrode merely by 50%. However, considering the influences of the 50% radius increase on the ablation volume, the ablation and necrosis zone was remarkably enlarged.

Although the preferred embodiments of the present invention have been described, it is understood that the present invention should not be limited to these preferred embodiments but various changes and modifications can be made by one skilled in the art within the spirit and scope of the present invention as hereinafter claimed.

1. An electrode for an electro-surgical operation device, comprising:
a hollow electrode formed in a hollow tube shape extending from a closed tip;
a first non-insulation area formed to a predetermined length from the closed tip;
a first insulation area formed on an outside surface of the hollow electrode beginning at the predetermined length from the closed tip;
a refrigerant tube, having a smaller diameter than a diameter of the hollow electrode, inserted into the hollow electrode, the refrigerant tube configured to supply refrigerants into the hollow electrode resulting to cool a living tissue in contact with the closed tip and/or the hollow electrode, and further configured to externally discharge heat-exchanged refrigerants from the living tissue through a gap between the refrigerant tube and the hollow electrode;
at least one first hole formed on the outside surface of the first non-insulation area;
the first hole operable to externally discharge a portion of the refrigerants supplied through the refrigerant tube into the living tissue in contact with the closed tip and/or the hollow electrode; and
a flow control mechanism formed on the outside surface of the first non-insulation area, and operable to act as a discharge resistance to the refrigerants discharged from the first hole, so as to control a flow of the refrigerants.

2. (canceled)

3. The electrode of claim 1, further comprising:
a saline solution pipe inserted onto the outside surface of the hollow electrode with a predetermined gap, and having a second non-insulation area at another predetermined length toward the closed tip and a second insulation area on the outside surface except the second non-insulation area;
the saline solution pipe operable to infuse a saline solution through the gap, and discharge the saline solution through at least one second hole formed on the outside surface of the second non-insulation area.

4. The electrode of claim 3, wherein the hollow electrode and the saline solution pipe are conductive, further comprising:
a power source operable to apply different power to the hollow electrode and the saline solution pipe; and
an insulation member formed on the surface of the hollow electrode and configured to prevent short circuit of the saline solution supplied through the gap between the hollow electrode and the saline solution pipe.

5. The electrode of claim 4, wherein the insulation member comprises the first insulation area formed on the surface of the hollow electrode, and an insulation packing provided between the hollow electrode and the saline solution pipe.

6. The electrode of claim 1, wherein the closed tip of the hollow electrode is a conductive spear head, and the hollow electrode and the spearhead are incorporated with each other.

7. The electrode of claim 1, wherein the flow control mechanism is a hollow tube inserted onto the outside surface of the first non-insulation area, and having a third hole on the outside surface, the flow control mechanism controlling a volume of the discharged refrigerants by alternately installing the first hole of the hollow electrode and the third hole of the hollow tube and operating as a discharge resistance to the refrigerants discharged from the first hole.

8. The electrode of claim 7, wherein compression units of the hollow tube are formed in a zigzag shape on a discharge passage from the first hole to the third hole, and operated as discharge resistances to the refrigerants discharged from the first hole, so as to control the volume of the discharged refrigerants.

9. The electrode of claim 1, wherein the flow control mechanism is a porous metal sintered body layer formed on the outside surface of the first non-insulation area;
the sintered body layer operable to act as discharge resistance to the refrigerants discharged from the first hole, so as to control the volume of the discharged refrigerants.

10. (canceled)

11. (canceled)

12. (canceled)

13. (canceled)

14. (canceled)

15. The electrode of claim 3, wherein the flow control mechanism is a hollow tube inserted onto the outside surface of the first non-insulation area, and having a third hole on the outside surface, the flow control mechanism controlling a volume of the discharged refrigerants by alternately installing the first hole of the hollow electrode and the third hole of the hollow tube and operating as a discharge resistance to the refrigerants discharged from the first hole.

16. The electrode of claim 4, wherein the flow control mechanism is a hollow tube inserted onto the outside surface of the first non-insulation area, and having a third hole on the outside surface, the flow control mechanism controlling a volume of the discharged refrigerants by alternately installing the first hole of the hollow electrode and the third hole of the hollow tube and operating as a discharge resistance to the refrigerants discharged from the first hole.

17. The electrode of claim 5, wherein the flow control mechanism is a hollow tube inserted onto the outside surface of the first non-insulation area, and having a third hole on the outside surface, the flow control mechanism controlling a volume of the discharged refrigerants by alternately installing the first hole of the hollow electrode and the third hole of the hollow tube and operating as a discharge resistance to the refrigerants discharged from the first hole.

18. (canceled)

19. (canceled)

20. (canceled)

21. An electrode for an electro-surgical operation device, comprising:
a hollow electrode formed in a hollow tube shape extending from a closed tip;
a first non-insulation area formed to a predetermined length from the closed tip;
a first insulation area formed on an outside surface of the hollow electrode beginning at the predetermined length from the closed tip;
a refrigerant tube, having a smaller diameter than a diameter of the hollow electrode, inserted into the hollow electrode, the refrigerant tube configured to supply refrigerants into the hollow electrode resulting to cool a living tissue in contact with the closed tip and/or the hollow electrode, and further configured to externally discharge heat-exchanged refrigerants from the living tissue through a gap between the refrigerant tube and the hollow electrode; and
a refrigerant discharging mechanism formed in the first non-insulation area, operable to externally discharge a portion of the refrigerants supplied through the refrigerant tube into the living tissue in contact with the closed tip and/or the hollow electrode.

22. The electrode of claim 21, further comprising:
a saline solution pipe inserted onto the outside surface of the hollow electrode with a predetermined gap, and having a second non-insulation area at another predetermined length toward the closed tip and a second insulation area on the outside surface except the second non-insulation area;
the saline solution pipe operable to infuse a saline solution through the gap, and discharge the saline solution through at least one second hole formed on the outside surface of the second non-insulation area.

23. The electrode of claim 22, wherein the hollow electrode and the saline solution pipe are conductive, further comprising:
a power source operable to apply different power to the hollow electrode and the saline solution pipe; and
an insulation member formed on the surface of the hollow electrode and configured to prevent short circuit of the saline solution supplied through the gap between the hollow electrode and the saline solution pipe.

24. The electrode of claim 4, wherein the insulation member comprises the first insulation area formed on the surface of the hollow electrode, and an insulation packing provided between the hollow electrode and the saline solution pipe.

25. The electrode of claim 21, wherein the refrigerant discharging mechanism is a porous metal sintered body formed in the first non-insulation area;
the sintered body operable to act a discharge resistance to the refrigerants supplied through the refrigerant tube, so as to control the volume of the discharged refrigerants.

26. A method for an electro-surgical operation comprising:
inserting an ablation device including at least one electrode into a wanted region in a living body; and
proceeding a radio-frequency ablation at the wanted region, when relatively much refrigerant is supplied from the outside into the electrode so as to cool a living tissue in contact with the electrode and relatively little refrigerant discharges into the living tissue.

27. The method of claim 26, wherein the discharged refrigerant is a portion of refrigerant supplied so as to cool the living tissue.

28. The method of claim 26, wherein the discharged refrigerant is supplied into the living tissue through at least one different channel from a channel for supplying the refrigerant so as to cool the living tissue.
